(19) United States
(12) Patent Application Publication
(10) Pub. No.: US 2019/0128885 A1
(43) Pub. Date: May 2, 2019

(54) SELECTION OF PANCREATIC PROGENITORS

(71) Applicant: UNIVERSITY HEALTH NETWORK, Toronto (CA)

(72) Inventors: Maria Cristina NOSTRO, Toronto (CA); Thomas KISLINGER, Toronto (CA); Kathryn COGGER, Toronto (CA); Ankit SINHA, Toronto (CA); Farida SARANGI, Toronto (CA)

(21) Appl. No.: 16/043,782

(22) Filed: Jul. 24, 2018

Related U.S. Application Data
(60) Provisional application No. 62/536,615, filed on Jul. 25, 2017.

(51) Int. Cl. G01N 33/566; C12N 5/071

(52) U.S. Cl. CPC G01N 33/566; C12N 5/0678

(57) ABSTRACT

There is described herein a method for enriching/purifying a population of cells for pancreatic progenitor cells, the method comprising: a) providing the population cells, the population comprising pancreatic progenitor cells; and b) performing at least one of steps (i)-(v): (i) selecting for cells from the population that express at least one protein listed in cluster 2; (ii) selecting for cells from the population that express at least one protein listed in cluster 5; (iii) deselecting for cells from the population that express at least one protein listed in cluster 1; (iv) deselecting for cells from the population that express at least one protein listed in cluster 3; and (v) deselecting for cells from the population that express at least one protein listed in cluster 6.

### RELATED APPLICATION

This application claims priority to U.S. Provisional Application No. 62/536,615 filed on Jul. 25, 2017, which is incorporated herein in its entirety.

### FIELD OF THE INVENTION

The invention relates to the selection of pancreatic progenitors using panels/cluster of protein markers.

### BACKGROUND OF THE INVENTION

Exogenous insulin administration to individuals with type 1 diabetes (T1d) is a life-saving therapy, but does not mimic the fine-tuned blood glucose control achieved by insulin secretion from endogenous pancreatic islet β cells¹. The success of whole pancreas and especially islet transplantation has provided compelling evidence that β cell-replacement therapy is a promising alternative treatment option for T1d, however the shortage of organ donors and required life-long immunosuppressive regimen limit their widespread use¹. In contrast, hPSCs could provide an unlimited supply of insulin-producing cells, and together with immunoprotective or tolerogenic strategies could restore endogenous insulin secretion in patients with T1d and selected type 2 diabetics²⁻⁵. Differentiation protocols designed to mimic pancreatic organogenesis in vitro have been successfully used to generate hPSC-derived PPs. These PPs express PDX1 and NKX6-1, both markers of pancreatic progenitors, and have the potential to give rise to insulin-producing cells in vivo and in vitro⁶⁻¹⁴. While human embryonic stem cell (hESC)-derived PPs are currently being tested for safety in a clinical trial for patients with T1d (NCT 02239354), protocol reproducibility across hPSC lines has been challenging even within the same laboratory, with the percentage of hPSC-derived PPs ranging from 6-45%⁷ and 36-83%⁹.

### SUMMARY OF THE INVENTION

In an aspect, there is provided a method for enriching/purifying a population of cells for pancreatic progenitor cells, the method comprising: a) providing the population cells, the population comprising pancreatic progenitor cells; and b) performing at least one of steps (i)-(iv): (i) selecting for cells from the population that express at least one protein listed in cluster 2; (ii) selecting for cells from the population that express at least one protein listed in cluster 5; (iii) deselecting for cells from the population that express at least one protein listed in cluster 1; (iv) deselecting for cells from the population that express at least one protein listed in cluster 3; and (v) deselecting for cells from the population that express at least one protein listed in cluster 6.

In an aspect, there is provided a kit comprising a plurality of antibodies against a plurality of proteins from at least one of cluster 1, 2, 3, 5, 6 and combinations thereof.

### BRIEF DESCRIPTION OF FIGURES

These and other features of the preferred embodiments of the invention will become more apparent in the following detailed description in which reference is made to the appended drawings wherein:

FIGS. 1A and 1B show membrane proteome analysis of undifferentiated hESCs, and day 13 differentiated PP and PH cultures. FIG. 1A: Schematic representation of the 4 stages of differentiation from human embryonic stem cells (hESCs) to pancreatic progenitor (PP) or polyhormonal (PH) cells. FIG. 1B: Line graphs showing the intensity profiles of deglycosylated sites for the first six clusters. The bold line represents the mean intensity profile of all the deglycosylated sites in the respective cluster.

FIGS. 2A, 2B and 2C show validation of the PP marker GP2 by flow cytometry, qPCR, and immunocytochemistry. FIGS. 1A and 1B: Flow cytometry analyses of undifferentiated hESCs, and day 13 PP and PH cultures. Cells were stained with anti-GP2. N=11 for hESC, N=9 for PP and N=8 for PH, error bars indicate s.e.m. ***p<0.001. FIG. 1C: qPCR analyses of GP2 in undifferentiated hESCs, and day 13 PP and PH cultures. Expression levels normalized to TBP, and relative to adult pancreas (equal to 1, not shown). N=3 for hESC and N=4 for PP and PH, error bars indicate s.e.m. *p<0.05, **p<0.01.

FIGS. 3A, 3B and 3C show FACS- and MACS-sorted GP2′ cells give rise to ‘β-like’ cells in vitro. FIG. 1A: Representative flow cytometry plots of day 23 cultures from H1-derived unsorted (presort), GP2⁺ or GP2⁻ populations stained with anti-NKX6-1 and anti-C-PEPTIDE (CPEP) antibodies. The bar graph shows the average percentage of double positive NKX6-1⁺/C-PEPTIDE⁺ cells. N=5, error bars indicate s.e.m. *p<0.05. FIGS. 1B and 1C: Representative flow cytometry plots of day 23 cultures from H9-derived cells FIG. 1B: and BJ-1-hlPSC FIG. 1C: following MACS sorting for GP2 at day 13, cells were cultured to day 23 in a modified version of the previously described protocols^{10,11}. Representative flow cytometry plots of NKX6-1 and C-PEPTIDE (CPEP) expression at day 23 of differentiation from either unsorted (PRESORT), enriched for GP2 using a MACS positive selection column (GP2⁺) or in the flow through cell population (Flow-). The bar graph shows the average percentage of double positive NKX6-1⁺/C-PEPTIDE⁺ cells at Day 23. N=4, error bars indicate s.e.m. **p<0.01.

### DETAILED DESCRIPTION

In the following description, numerous specific details are set forth to provide a thorough understanding of the invention. However, it is understood that the invention may be practiced without these specific details.

PDX1⁺/NKX6-1⁺ pancreatic progenitors (PPs) can be successfully differentiated from human Pluripotent Stem Cells (hPSCs) and hold the potential to generate an unlimited supply of β-cells for diabetes treatment. However, the efficiency of PP generation in vitro is highly variable, negatively impacting reproducibility and validation of in vitro and in vivo studies, and consequently, translation to the clinic. A proteomics approach was used to phenotypically characterize hPSC-derived PPs and distinguish these cells from non-PP populations during differentiation. The analysis identified panels/cluster of proteins as PP-specific cell surface markers. For example, pancreatic secretory granule membrane major glycoprotein 2 (GP2) was identified. Isolated hPSC-derived GP2⁺ cells generate β-like cells (C-PEPTIDE⁺/NKX6-1⁺) more efficiently compared to GP2⁻ and unsorted populations, underlining the potential therapeutic applications of markers such as GP2. Furthermore, clusters of protein markers were also identified for use as a negative selector.

In an aspect, there is provided a method for enriching/purifying a population of cells for pancreatic progenitor cells, the method comprising: a) providing the population cells, the population comprising pancreatic progenitor cells; and b) performing at least one of steps (i)-(iv): (i) selecting for cells from the population that express at least one protein listed in cluster 2; (ii) selecting for cells from the population that express at least one protein listed in cluster 5; (iii) deselecting for cells from the population that express at least one protein listed in cluster 1; (iv) deselecting for cells from the population that express at least one protein listed in cluster 3; and (v) deselecting for cells from the population that express at least one protein listed in cluster 6.

Methods of differentiating stem cell populations into pancreatic progenitors are known to a person skilled in the art, as exemplified in reference 20-25 cited herein. Similarly, identification, characterization and definition of pancreatic progenitors are also known to a person skilled in the art as exemplified in reference 15 cited herein

In some embodiments, the at least one protein listed in cluster 2 is any number from 2 to u, wherein u is the total number of proteins listed in cluster 2.

In some embodiments, the at least one protein listed in cluster 5 is any number from 2 to w, wherein w is the total number of proteins listed in cluster 5.

In some embodiments, the at least one protein listed in cluster 1 is any number from 2 to x, wherein w is the total number of proteins listed in cluster 1.

In some embodiments, the at least one protein listed in cluster 3 is any number from 2 to y, wherein w is the total number of proteins listed in cluster 3.

In some embodiments, the at least one protein listed in cluster 6 is any number from 2 to z, wherein w is the total number of proteins listed in cluster 6.

In different embodiments, the at least one step is at least 2 steps, at least 3 steps, at least 4 steps, or at least 5 steps.

In some embodiments, the pancreatic progenitor cells are PDX1⁺/NKX6-1⁺.

In some embodiments, the population further comprises at least one of human embryonic stem cells, human pluripotent stem cells, and polyhormonal cells. The population may further comprise both human embryonic stem cells, and polyhormonal cells.

In some embodiments, the population had been induced to at least partially differentiate into pancreatic progenitor cells from human pluripotent stem cells.

In some embodiments, the selection or deselection step (i), (ii), (iii), (iv), or (v) are performed using an antibody against the at least one protein. Preferably, the antibody is bound to a fluorophore, and/or a support, preferably a magnetic bead.

Various cell sorting methods known in the art are capable of separating cells according to the surface protein expression. For example single cell sorting methods include an IsoRaft array and rhe DEPArray lab-on-a-chip technology. Other methods include Fluorescent Activated Cell Sorting which utilizes flow cytometry and Magnetic Cell Sorting, which includes Magnetic-activated cell sorting (MACS) and the SEP system.

In some embodiments, the selection or deselection step is performed using FACS.

In some embodiments, the selection or deselection step is performed using MACS.

In an aspect, there is provided a kit comprising a plurality of antibodies against a plurality of proteins from at least one of cluster 1, 2, 3, 6 and combinations thereof.

In an embodiment, the plurality of antibodies in the kit are bound to magnetic beads.

In some embodiments, the plurality of proteins is any number between 2 and q, wherein q is independently the total number of proteins listed in a particular cluster

The advantages of the present invention are further illustrated by the following examples. The examples and their particular details set forth herein are presented for illustration only and should not be construed as a limitation on the claims of the present invention.

### EXAMPLES

Methods and Materials

Culture and Differentiation of hESCs

H1 and H9 hESCs were obtained from WiCell; NKX6-1^{GFP/w} hESCs were provided by Drs. Stanley and Elefanty⁹. BJ-iPSC1 was provided by Drs. Araki and Neel³⁰. Undifferentiated hESCs tested negative for mycoplasma and were maintained as previously described³¹. Differentiation was initiated when the hESC cultures reached 70-80% confluence. As described previously¹⁵, monolayer cultures were treated with RPMI (Gibco) containing 100 ng/ml hActivin A (R&D Systems) and 2 μM CHIR990210 (Tocris) for one day (d0-d1). They were then cultured for two days in RPMI media containing 100 ng/ml hActivin A and 5 ng/ml hbFGF (R&D Systems) (d1-d3). This completed stage 1 of differentiation. During stage 2, cells were cultured in RPMI with 1% vol/vol B27 supplement (without vitamin A) (Life Technologies), 50 ng/ml hFGF10 (R&D Systems), 0.75 μM dorsomorphin (Sigma), and 3 ng/ml mWnt3a (R&D Systems) (d3-d6). On day 6 of differentiation, cultures were transferred to stage 3 media, consisting of DMEM (Gibco) with 1% vol/vol B27 supplement, 50 μg/ml ascorbic acid (Sigma), 50 ng/ml hNOGGIN (R&D Systems), 50 ng/ml hFGF10, 0.25 μM SANT-1 (Tocris), and 2 μM all-trans RA (Sigma), and cultured for 2-3 days. During stage 4, the media was changed to DMEM containing 1% vol/vol B27 supplement, 50 μg/ml ascorbic acid and 50 ng/ml hNOGGIN, and supplemented with either 100 ng/ml hEGF (R&D Systems) and 10 mM nicotinamide (Sigma), to direct cells towards the PP lineage (d8-d13), or 6 μM SB431542 (Sigma) to generate PH cells (d9-d13). H1 cells for FACS sorting received media supplemented with 100 ng/ml hEGF and 3.3 mM nicotinamide at stage 4 to obtain a GP2-low expressing population. H9 cells received stage 3 media for 3 days, and received media supplemented with 50 ng/ml hEGF and 10 mM nicotinamide at stage 4, as previously described⁹. BJ-iPSC1 cells received stage 3 media for 1 day, and received media supplemented with 100 ng/ml hEGF and 10 mM nicotinamide at stage 4, as previously described⁹.

For stage 5 and 6, single cells obtained after FAC- and MAC-sorting were cultured in suspension at 2×10⁶ cells/ml in low-adherent tissue culture plates in MCDB131 media (Gibco) containing 1 μM T3 (Sigma), 1.5 g/L NaHCO₃ (Gibco), 1% vol/vol L-Glutamine (GE Healthcare), 1% vol/vol B27 supplement (Gibco), 15 mM D-(+)-Glucose (Sigma), 10 μg/ml Heparin (Sigma), 0.25 μM SANT-1 (Tocris), 10 μM RepSox (Tocris), 100 nM LDN193189 (Cayman), 10 μM ZnSO₄ (Sigma), 0.05 μM all-trans RA (Sigma), and 10 μM Y27632 (Tocris) (d13-d16, stage 5). After 72 hours, the media was changed to MCDB131 containing 1 μM T3, 1.5 g/L NaHCO₃, 1% vol/vol L-Glutamine, 1% vol/vol B27 supplement, 15 mM D-(+)-Glucose, 10 μg/ml Heparin, 10 μM RepSox, 100 nM LDN193189, 10 μM ZnSO₄, and 100 nM DBZ (Tocris) (d16-d23, stage 6), media was replenished at day 19. Aggregates were harvested at day 23, dissociated with trypsin for flow cytometry or fixed in 1.6% PFA for immunohistochemistry analysis. Stage 5 and stage 6 media components are based on the previously described protocols^{10,11}.

Mass Spectrometry

Digestion of Proteins for MS-Based Proteomics

Obtained cell pellets were resuspended in 50% (vol/vol) 2,2,2-trifluoroethanol in phosphate buffer saline (pH 7.4) and 10 pmol of invertase protein (SUC2, a yeast glyco-protein, Sigma-Aldrich; UniProt accession—P00724) was spiked-in each sample as an internal method control. Cell lysis was induced through pulse sonication. Subsequently, lysates were incubated at 60° C. for 2 hours with brief agitation every 30 minutes. The cysteines in the protein lysates were reduced through the addition of 5 mM dithiothreitol and incubated at 60° C. for 30 minutes. The lysates were cooled prior to alkylation of reduced cysteines, which was performed with 25 mM iodoacetamide at room temperature for 30 minutes in the dark. Subsequently, all samples were diluted 1:5 (v/v) using 100 mM ammonium bicarbonate (pH 8.0). Mass-spectrometry grade trypsin (trypsin:protein ratio 1:50, Promega) was added and digestion was performed at 37° C. for 16 hours. Peptides were desalted using C18 MacroSpin Columns (Nest Group) and lyophilized using a vacuum concentrator.

Enrichment of N-Glycosylated Peptides

Solid Phase extraction of N-glycosylated Peptides (SPEG) strategy was used for enriching N-glycosylated peptides from a pool of peptides. Briefly, lyophilized peptides were re-suspended in 100 mM sodium acetate/150 mM sodium chloride at pH 5.5. Glycans present on peptides were oxidized by adding 10 mM sodium meta-periodate and incubated at room temperature for 30 minutes in the dark. Peptides were desalted, lyophilized and solubilized in the sodium acetate buffer. Peptides with oxidized-glycans were captured on hydrazide labeled magnetic beads (glycoprotein:bead ratio 1:1, Chemicell) for 16 hours with constant rotation. The supernatant containing unbound peptides was aspirated, and the beads were washed with 1.5 M sodium chloride, water, methanol, acetonitrile and 100 mM ammonium bicarbonate at pH 8.0. Bound peptides were eluted from the beads using PNGaseF (Roche), and incubated at 37° C. for 16 hours. The supernatant containing the deglycosylated peptides was desalted, lyophilized and measured for peptide concentration using the Thermo Scientific Nanodrop 2000 spectrophotometer.

Mass Spectrometry and Peptide/Protein Identification

1.5 μg of deglycosylated peptides were injected for each LC-MS/MS analysis, as described previously³³. Briefly, peptides were separated using reverse-phase chromatography with a 4 hour gradient. Chromatography was performed using a 50 cm column with a flow rate of 250 nL/min using the Thermo Scientific EasyLC1000 nano-liquid-chromatography system. QExactive tandem mass spectrometry was used for acquiring MS/MS data while operating in a data dependent mode.

Protein Identification from the Mass Spectrometry Data

The acquired raw-files were analyzed with MaxQuant (version: 1.5.0.0) using UniProt complete human proteome protein sequence database (version: 2012-07-19, number of sequences: 20,232)³⁴. Searches were performed with a maximum of two missed cleavages, carbamidomethylation of cysteine was specified as a fixed modification, and oxidation of methionine and deamidation of asparagine to aspartic acid were specified as variable modifications. False discovery of peptides was controlled using a target-decoy approach based on reverted sequences, and the false discovery rate was defined as 1% at site, peptide, and protein levels.

Data Analysis

All analysis was based on the data present in the Maxquant output file named Asn-_AspSites.txt and performed using R. All deamidated sites containing either SER or THR at the +2 site of the deamidated-ASN were carried forward for analysis, and were defined as deglycosylated sites. For each cell type, a deglycosylated site was considered to be present if it was quantified in at least two replicates. All gene ontology, pathway and protein keyword analysis was performed using ProteinCenter (Thermo Scientific). Unsupervised clustering of samples was performed using Pearson correlation, and K-means unsupervised learning algorithm was used for clustering deglycosylated sites into 11 clusters (determined using scree plot of sum of squared error) based on their intensity profile in the three biological samples.

Flow Cytometry and Fluorescence-Activated Cell Sorting (FACS)

Cells were dissociated from the monolayer using either TrypLE Express (Gibco) or StemPro Accutase (Gibco) and incubated at 37° C. to generate a single-cell suspension. Live cells were incubated for 20 minutes at 4° C. with primary antibodies in 1×PBS/10% fetal bovine serum (FBS; Wisent Inc.) (FACS buffer). After washing twice with FACS buffer, samples labeled with unconjugated primary antibodies were incubated for an additional 20 minutes at 4° C. with secondary antibodies in FACS buffer.

For FACS, cells were processed as above, and re-suspended in 1×PBS/0.1% FBS. They were sorted using the BD AriaII-RITT or AriaIII Fusion cell sorters.

For all intracellular staining, except PDX1, cells were fixed in 1.6% paraformaldehyde (PFA) for 24 hours at 4° C. Samples were then washed twice in FACS buffer, and incubated overnight at 4° C. with primary antibodies in 1×PBS containing 5 mg/ml saponin (Sigma) (saponin). After two washes in FACS buffer, the cells were then incubated with secondary antibodies in saponin for 30-45 minutes at room temperature. For PDX1 intracellular staining, cells were fixed in BD Bioscience cytofix/cytoperm buffer (Cat.#554722) for 24 hours at 4° C. They were then washed twice in 1×BD Bioscience Perm/Wash (Cat.#554723) and incubated with anti-PDX1 primary antibody (1/100, R&D Systems AF2419) in BD Bioscience Perm/Wash, for 1 hour at room temperature. Following 2 washes in the BD Bioscience Perm/Wash, a donkey α goat AF647 or donkey α goat AF488 secondary antibody (Jackson ImmunoResearch Laboratories Inc. 705-606-147 or 705-546-147) diluted 1/400 in Perm/Wash was applied, and the cells incubated again for 1 hour at room temperature.

For the triple staining GP2-PDX1-NKX6-1 and CD142-PDX1-NKX6-1, live cells were incubated for 20 minutes at 4° C. with an anti-GP2 antibody or anti-CD142 antibody in 1×PBS/10% fetal bovine serum (FBS; Wisent Inc.) (FACS buffer). After washing once with FACS buffer, the live cells were incubated for an additional 20 minutes at 4° C. with an anti-mouse PE-conjugated secondary antibody in FACS buffer. The cells were fixed in BD Bioscience cytofix/cytoperm buffer (Cat.#554722) for 24 hours at 4° C.

They were then washed twice in 1×BD Bioscience Perm/Wash (Cat.#554723) and incubated with anti-PDX1 primary antibody (1/100, R&D Systems AF2419) and anti-NKX6-1 primary antibody (1/2000, Developmental Studies Hybridoma Bank F55A10) in BD Bioscience Perm/Wash, for 1 hour at room temperature. Following 2 washes in the BD Bioscience Perm/Wash, donkey anti goat AF488 secondary antibody (Jackson ImmunoResearch Laboratories Inc. 705-546-147) and donkey anti mouse AF647 (Life Technology A31571) diluted 1/400 in Perm/Wash was applied, and the cells incubated again for 1 hour at room temperature. Mouse, rat and goat IgGs were used as control. Flow cytometry was carried out using the BD LSR Fortessa flow cytometer, and data were analyzed using FlowJo software. In addition to the anti-GP2 antibody, two additional antibodies, HPx1 and HPx2 (Novus Biologicals, Littleton, Colo.) raised against human exocrine pancreas²² were used to track GP2⁺ cells.

Magnetic Activated Cell Sorting (MACS)

Cells were dissociated from the monolayer using TrypLE Express (Gibco) and washed 1× in PBS/10% fetal bovine serum (FBS; Wisent Inc.) (FACS buffer) containing 10 μM Y27632 (Tocris). Live cells were incubated for 20 minutes at at room temperature with an anti-GP2 antibody in FACS buffer. After washing once with FACS buffer, the live cells were incubated for an additional 20 minutes at at room temperature with an anti-mouse PE-conjugated secondary antibody in FACS buffer. The cells were washed once again with FACS buffer, and resuspended in MACS buffer (Miltenyi Biotec) with anti-PE microbeads (Miltenyi Biotec) at a concentration of 75 μl MACS buffer and 20 μl beads/1×10⁷ cells. They were incubated at 4° C. for 15 minutes, followed by one wash in MACS buffer. The cells were then loaded onto a LS positive selection column (Miltenyi Biotec) and the flow through collected. The column was washed thrice and the cells eluted, all in FACS buffer with 10 μM Y27632 (Tocris). The elution from the LS positive selection column formed the GP2⁺ fraction. The flow through from the LS positive selection column was then loaded onto a LD depletion column (Miltenyi Biotec). The flow through from the LD depletion column was used as the flow through fraction. Following MACS, the cells were cultured as aggregates.

Quantitative PCR

The Ambion PureLink RNA mini kit was used to extract total cellular RNA. cDNA reverse transcription was then conducted using Superscript III reverse transcriptase and RNAseOUT recombinant ribonuclease inhibitor (Invitrogen). qPCR was performed using BioRad SsoAdvanced SYBR green supermix and the Biorad CFX Connect real-time system. Relative gene expression was normalized to the housekeeping gene TBP, and fold-change calculated based on a comparison to the expression level of the adult pancreas. Adult Pancreas total RNA was purchased from Takara (Cat #636577, Lot #1202351A).

Immunostaining

For live staining of GP2, monolayer cultures were dissociated to single-cells using TrypLE Express (Gibco) at 37° C., and stained as described for flow cytometry. The labeled cells were then re-suspended at a concentration of 100,000 cells/ml in 1×PBS/10% FBS (Wisent Inc.). 100 μl of cell suspension was used per slide, and centrifuged at 85 RCF in the Thermo Fisher Scientific Shandon Cytospin 4. The slides were then fixed in 1.6% PFA for 24 hours at 4° C.

hESC-derived, GP2-sorted aggregates were harvested at day 23 of differentiation and fixed in 1.6% PFA for 48 hours at 4° C. They were then embedded in agarose and paraffin, and 3 μm sections were cut by the Toronto General Hospital, Pathology Research Program Laboratory. Sections were de-paraffinized using xylene, and rehydrated in a serial dilution of absolute alcohol. Antigen-retrieval was performed, and the sections were blocked using 10% non-immune donkey serum (Jackson ImmunoResearch Laboratories Inc.) in PBS. Primary antibodies were diluted in 1×PBS supplemented with 0.3% Triton X-100 (Sigma) and 0.25% BSA (Sigma) (PBS-Triton-BSA), and incubation was conducted at 4° C. overnight. After washing, the sections were incubated with secondary antibodies in PBS-Triton-BSA for 45 minutes at room temperature.

Cryosections of human pancreatic tissue obtained through the Neonatal Donor Program of the International Institute for the Advancement of Medicine (IIAM) were post-fixed with 1% PFA for 10 minutes and permeabilized with 0.5% Triton X-100/1×PBS for 15 minutes at room temperature. The sections were then blocked with 5% normal donkey serum (Sigma)/1×PBS and incubated overnight at 4° C. with primary antibodies diluted in 1×PBS supplemented with 0.2% Triton X-100 and 1% BSA (PBS-Triton-BSA). After washing, the sections were incubated with secondary antibodies in PBS-Triton-BSA for 2 hours at room temperature.

Slides were counterstained with DAPI (Biotium) for 1 minute, and mounted with Dako fluorescent mounting media. Digital images were acquired using the Zeiss LSM700 and LSM510 META laser scanning confocal microscopes and Zen confocal software, using 20×, 40× and 63× objectives. Digital images were acquired using a Leica DM16000B fluorescence microscope equipped with a Leica DFC360FX digital camera. Digital images were acquired using the EVOS FL Cell Imaging System (ThermoFisher), using 40×.

Statistical Analysis

All flow cytometry, and qPCR data were analyzed using an unpaired Student's t-test.

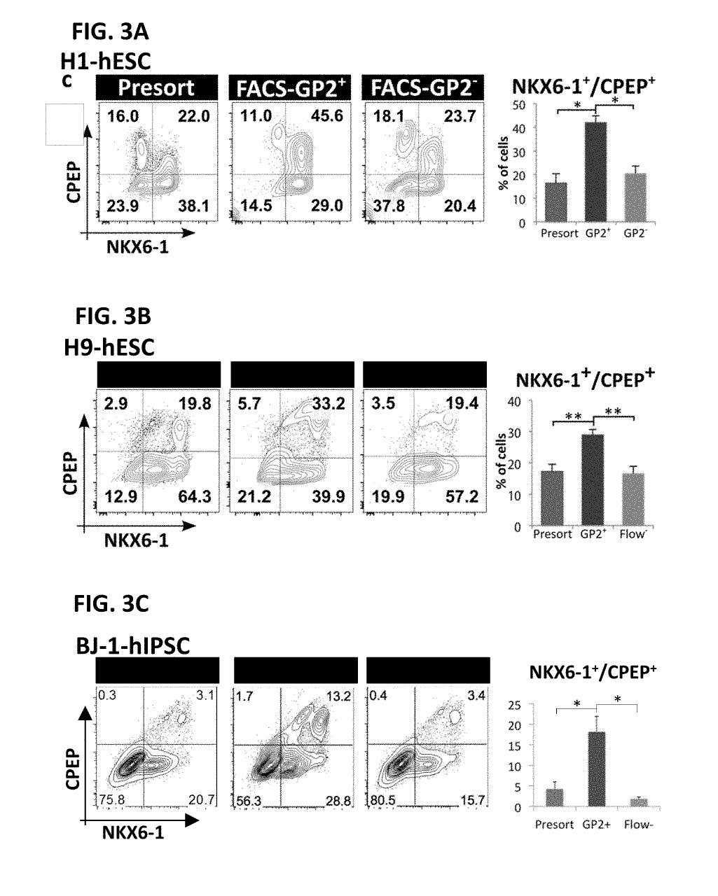

One-way ANOVA followed by Tukey's multiple comparisons test was used to analyze the percentage of C-Peptide⁺/NKX6-1⁺ cells generated using H1, H9 and BJ-iPSC1 cells (FIG. 3a, 3b-c).

Results and Discussion

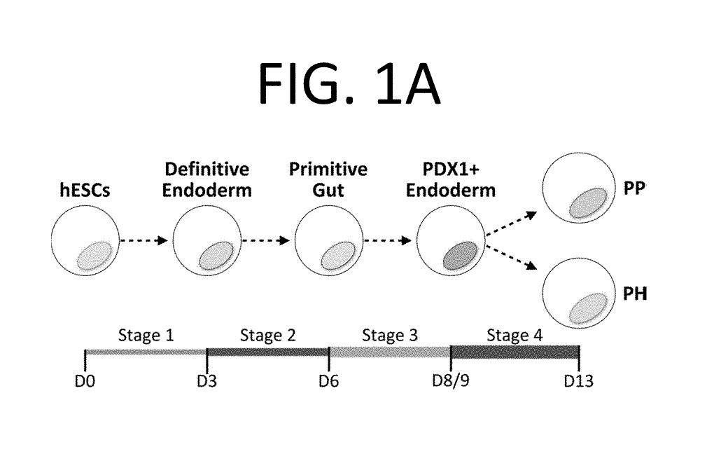

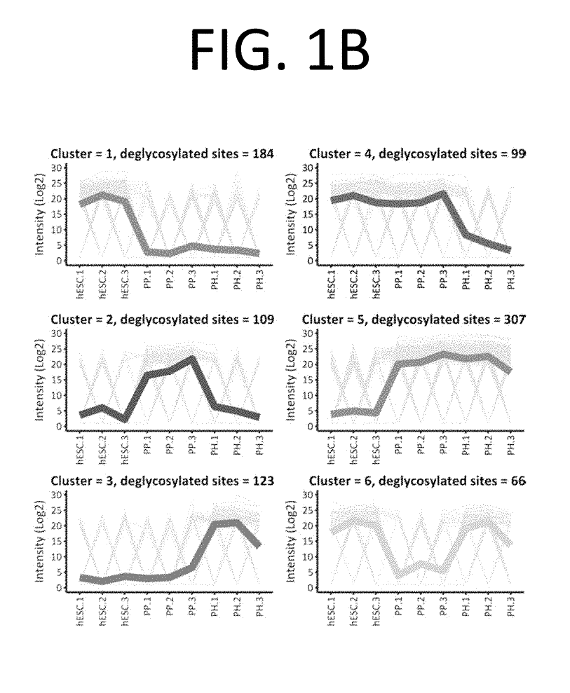

To provide a safer cell population for therapeutic purposes and obviate the risk of contamination from undifferentiated hPSCs and/or other germ layer derivatives, hESCs were differentiated towards two independent pancreatic populations: PPs and polyhormonal (PH) cells using two distinct differentiation protocols¹⁵ (FIG. 1a). The PP population is primarily composed of PDX1⁺/NKX6-1⁺ cells (>80%) and shows no detectable expression of associated mature endocrine (INS, GCG) and pluripotency (OCT4, SOX2) genes. Upon transplantation beneath the mouse kidney capsule, PPs are able to generate all lineages of the pancreas including β-cells^{7-9,12,16}. In contrast, the PH differentiation protocol generates fewer NKX6-1⁺ cells (10.5%±1.1%) and a higher percentage of CPEP⁺/NKX6-1⁻ (10.0%±0.9%) and CPEP⁺/GCG⁺ (2.5%±0.3%) polyhormonal cells than the PP method. PH cells also express significantly lower levels of PDX1 and NKX6-1 compared to PPs and do not generate β-cells in vivo^{8,13,17}. Undifferentiated hESCs, along with the hESC-derived PP and PH cells were used for the selective enrichment of N-glycoproteins and compared by mass spectrometry. Characterization of the changes in the N-linked glycosylated proteome of hESC, PP and PH populations was performed using label-free shotgun proteomics of three independent replicates for each cell type. The analysis of the nine samples identified 2745 deamidation sites on asparagine residues, which mapped to 1043 protein groups.

Pearson correlation-based unsupervised clustering of the nine samples revealed a higher correlation coefficient between PP and PH cells (0.53) rather than with hESC, which is supported by the observation of a large number of shared proteins between these two cell populations. An internal control protein (yeast invertase—SUC2) confirmed data reproducibility and the absence of systematic bias. K-means based unsupervised clustering was used to partition the 2222 deglycosylated sites into 11 distinct clusters based on their intensity profiles. Cluster 1, 2 and 3 contain deglycosylated sites that were uniquely detected in hESC, PP or PH cells, respectively. The average intensity profiles for deglycosylated sites of the first six clusters are shown in FIG. 1b.

hESC reference markers such as ALPL, KDR and SOX2 were all enriched in the hESC-specific cluster. Of the 126 proteins listed in cluster 1, 23 belonged to a PSC-restricted list identified by previous proteomic studies¹⁹.

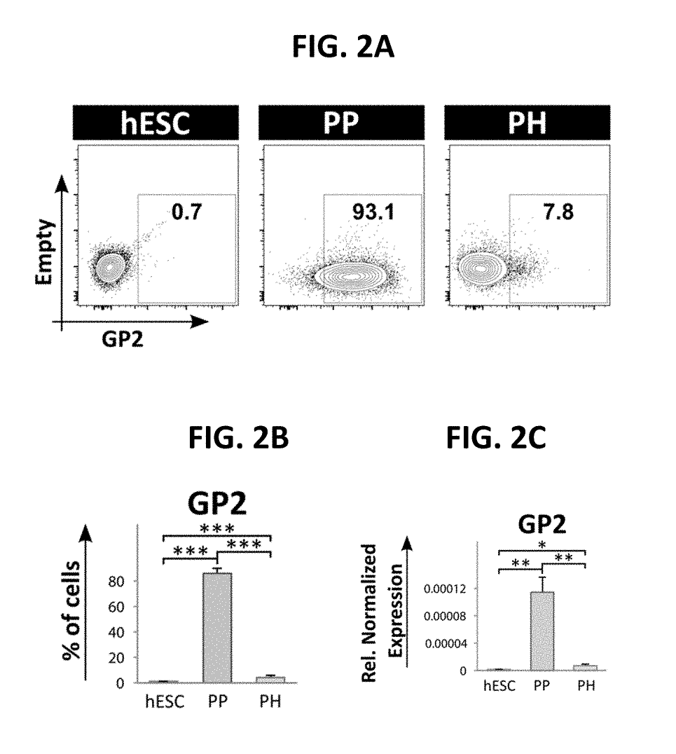

To identify novel markers of the PP population, proteins were systematically selected from clusters 2 and 5 (FIG. 1b) that presented the highest and most consistent changes in peptide intensity, and for which commercial antibodies were available for flow cytometry. Of the selected antibodies the pancreatic secretory granule membrane major glycoprotein 2 (GP2) was validated as an epitope that reliably and specifically marks the PPs, with over 85.0%±2.6% of cells expressing this marker by flow cytometry (FIG. 2a,b). GP2 expression levels were also significantly higher in the PP compared to undifferentiated hESC and PH cells, as assessed by qPCR (FIG. 2c).

To assess the use of GP2 as a cell surface marker to isolate pancreatic progenitor cells, fluorescence activated cell sorting (FACS) was used to isolate the GP2 positive from GP2 negative fractions at day 13 of differentiation using the H1 cell line. To ensure a sufficient pool of GP2⁻ cells, the amount of nicotinamide at stage 4 of differentiation was reduced to lower the differentiation efficiency and mimic the known variability that occurs with different PSC lines. Following a suboptimal differentiation, day 13 cultures containing 36% and 35% GP2⁺ and NKX6-1⁺ cells, respectively, were generated and using FACS, the top 25% of GP2⁺ cells, and a similar percentage of the GP2⁻ population, were isolated. To address whether the enriched GP2⁺ cells have an increased potential to generate β-like cells in vitro, the sorted and unsorted (presort) cells were cultured as aggregates in suspension, and assessed for their ability to give rise to NKX6-1⁺/CPEP⁺ cells^{10,11}. At day 23 of differentiation, cultures from GP2⁺ cells contained significantly more NKX6-1⁺/CPEP⁺ cells compared to GP2⁻ and unsorted cultures (FIG. 3a).

GP2 is identified as a specific marker of human pancreatic progenitors and describes an efficient strategy to purify these precursors from hPSCs. To validate the use of GP2 for translational purposes and to demonstrate efficient labeling of the population of interest, H9 was used, a cell line that could be used for clinical application (cGMP line distributed by WiCell) and that had previously shown substandard differentiation to the pancreatic lineage⁹. Day 13 H9-derived cultures contained a lower percentage of GP2⁺ cells compared to H1-derived cultures. H9-derived GP2⁺ cells were successfully isolated using MACS and cultured alongside cells from both the presort and flow through fractions. Similarly to the H1-fluorescence-activated cell sorted GP2⁺ population, the magnetic-activated sorted GP2⁺ cells gave rise to significantly more NKX6-1⁺/CPEP⁺ cells compared to flow through and unsorted populations (FIG. 3b-c). To verify that this method can be applied to additional cell lines, GP2⁺ cells were isolated using MACS from BJ-iPSC1, a human induced pluripotent stem cell line (hiPSC). BJ-iPSC1-derived GP2⁺ cells were successfully isolated using MACS and cultured alongside cells from both the presort and flow through fractions. Similar to the H1 and H9 sorted GP2⁺ populations, the magnetic activated cells sorted GP2⁺ cells gave rise to significantly more NKX6-1⁺/CPEP⁺ cells compared to flow through and unsorted populations.

A similar proteomic method was used to identify proteins that would be useful in identifying true pancreatic progenitor cells that produce insulin. Various clusters of proteins were thus identified. Cluster 1, 2 and 3 contain deglycosylated sites that were uniquely detected in hESC, PP or PH cells, respectively. Cluster 1 contains 127 proteins, cluster 2 contains 84 proteins and cluster 3 contains 87 proteins. Cluster 4 contains 99 deglycosylates sites detected in hESC and PP cells. These sites correspond to 76 proteins. Cluster 5 contains 307 deglycosylates sites detected in PP and PH cells. These sites correspond to 225 proteins. Cluster 6 contains 66 deglycosylates sites detected in hESC and PH cells. These sites correspond to 63 proteins. As such, these clusters may used to select for PP, positively or negatively, for example, by the use of antibodies against proteins in a cluster and flow cytometry.

The Clusters are provided below:

Cluster 1

A2RU67 (Uncharacterized protein KIAA1467), A2VDJ0 (Transmembrane protein 131-like), O14594 (Neurocan core protein), O15230 (Laminin subunit alpha-5), O60704 (Protein-tyrosine sulfotransferase 2), O75071 (EF-hand calcium-binding domain-containing protein 14), O75173 (A disintegrin and metalloproteinase with thrombospondin motifs 4), O75493 (Carbonic anhydrase-related protein 11), O75829 (Leukocyte cell-derived chemotaxin 1; Chondrosurfactant protein; Chondromodulin-1), O94898 (Leucine-rich repeats and immunoglobulin-like domains protein 2), O95196 (Chondroitin sulfate proteoglycan 5), O95206 (Protocadherin-8), O95450 (A disintegrin and metalloproteinase with thrombospondin motifs 2), O95490 (Latrophilin-2), O95672 (Endothelin-converting enzyme-like 1), P01137 (Transforming growth factor beta-1; Latency-associated peptide), P01903 (HLA class II histocompatibility antigen, DR alpha chain), P04035 (3-hydroxy-3-methylglutaryl-coenzyme A reductase), P05107 (Integrin beta-2), P05186 (Alkaline phosphatase, tissue-nonspecific isozyme), P06213 (Insulin receptor; Insulin receptor subunit alpha; Insulin receptor subunit beta), P07949 (Proto-oncogene tyrosine-protein kinase receptor Ret; Soluble RET kinase fragment; Extracellular cell-membrane anchored RET cadherin 120 kDa fragment), P08473 (Neprilysin), P12111 (Collagen alpha-3(VI) chain), P13611 (Versican core protein), P14415 (Sodium/potassium-transporting ATPase subunit beta-2), P17948 (Vascular endothelial growth factor receptor 1), P18084 (Integrin beta-5), P20908 (Collagen alpha-1(V) chain), P21579 (Synaptotagmin-1), P21757 (Macrophage scavenger receptor types I and II), P22303 (Acetylcholinesterase), P23327 (Sarcoplasmic reticulum histidine-rich calcium-binding protein), P23352 (Anosmin-1), P23471 (Receptor-type tyrosine-protein phosphatase zeta), P24043 (Laminin subunit alpha-2), P24821 (Tenascin), P25445 (Tumor necrosis factor receptor superfamily member 6), P25942 (Tumor necrosis factor receptor superfamily member 5), P27169 (Serum paraoxonase/arylesterase 1), P30532 (Neuronal acetylcholine receptor subunit alpha-5), P31644 (Gamma-aminobutyric acid receptor subunit alpha-5), P32942 (Intercellular adhesion molecule 3), P34903 (Gamma-aminobutyric acid receptor subunit alpha-3), P35968 (Vascular endothelial growth factor receptor 2), P37088 (Amiloride-sensitive sodium channel subunit alpha), P42785 (Lysosomal Pro-X carboxypeptidase), P45452 (Collagenase 3), P47972 (Neuronal pentraxin-2), P48431 (Transcription factor SOX-2), P51884 (Lumican), P61647 (Alpha-2,8-sialyltransferase 8F), P78357 (Contactin-associated protein 1), P98095 (Fibulin-2), Q02763 (Angiopoietin-1 receptor), Q04912 (Macrophage-stimulating protein receptor; Macrophage-stimulating protein receptor alpha chain; Macrophage-stimulating protein receptor beta chain), Q06430 (N-acetyllactosaminide beta-1,6-N-acetylglucosaminyl-transferase, isoform B), Q07092 (Collagen alpha-1(XVI) chain), Q07954 (Prolow-density lipoprotein receptor-related protein 1; Low-density lipoprotein receptor-related protein 1 85 kDa subunit; Low-density lipoprotein receptor-related protein 1 515 kDa subunit; Low-density lipoprotein receptor-related protein 1 intracellular domain), Q08174 (Protocadherin-1), Q10588 (ADP-ribosyl cyclase 2), Q10589 (Bone marrow stromal antigen 2), Q12770 (Sterol regulatory element-binding protein cleavage-activating protein), Q12860 (Contactin-1), Q12913 (Receptor-type tyrosine-protein phosphatase eta), Q13275 (Semaphorin-3F), Q13683 (Integrin alpha-7; Integrin alpha-7 heavy chain; Integrin alpha-7 light chain; Integrin alpha-7 70 kDa form), Q13753 (Laminin subunit gamma-2), Q14114 (Low-density lipoprotein receptor-related protein 8), Q14118 (Dystroglycan; Alpha-dystroglycan; Beta-dystroglycan), Q14766 (Latent-transforming growth factor beta-binding protein 1), Q15043 (Zinc transporter ZIP14), Q15818 (Neuronal pentraxin-1), Q30154 (HLA class II histocompatibility antigen, DR beta 5 chain), Q3T906 (N-acetylglucosamine-1-phosphotransferase subunits alpha/beta; N-acetylglucosamine-1-phosphotransferase subunit alpha; N-acetylglucosamine-1-phosphotransferase subunit beta), Q53EL9 (Seizure protein 6 homolog), Q5KU26 (Collectin-12), Q5VU97 (VWFA and cache domain-containing protein 1), Q6P4E1 (Protein CASC4), Q6P9A2 (Polypeptide N-acetylgalactosaminyltransferase 18), Q6UVK1 (Chondroitin sulfate proteoglycan 4), Q6UX71 (Plexin domain-containing protein 2), Q6UXZ4 (Netrin receptor UNC5D), Q6YHK3 (CD109 antigen), Q7LGC8 (Carbohydrate sulfotransferase 3), Q86YC3 (Leucine-rich repeat-containing protein 33), Q81WT1 (Sodium channel subunit beta-4), Q81WU6 (Extracellular sulfatase Sulf-1), Q8N0Z9 (V-set and immunoglobulin domain-containing protein 10), Q8NFM7 (Interleukin-17 receptor D), Q8WZ71 (Transmembrane protein 158), Q92673 (Sortilin-related receptor), Q92820 (Gamma-glutamyl hydrolase), Q92859 (Neogenin), Q96AM1 (Mas-related G-protein coupled receptor member F), Q96FE5 (Leucine-rich repeat and immunoglobulin-like domain-containing nogo receptor-interacting protein 1), Q96JP9 (Cadherin-related family member 1), Q96MK3 (Protein FAM20A), Q96NR3 (Patched domain-containing protein 1), Q9BXJ5 (Complement Clq tumor necrosis factor-related protein 2), Q9GZX3 (Carbohydrate sulfotransferase 6), Q9GZX9 (Twisted gastrulation protein homolog 1), Q9H2E6 (Semaphorin-6A), Q9H4B8 (Dipeptidase 3), Q9H6B4 (CXADR-like membrane protein), Q9H9K5 (HERV-MER_4q12 provirus ancestral Env polyprotein), Q9HB40 (Retinoid-inducible serine carboxypeptidase), Q9HCQ5 (Polypeptide N-acetylgalactosaminyltransferase 9), Q9NPC4 (Lactosylceramide 4-alpha-galactosyltransferase), Q9NS84 (Carbohydrate sulfotransferase 7), Q9NXG6 (Transmembrane prolyl 4-hydroxylase), Q9NY97 (UDP-GlcNAc:betaGal beta-1,3-N-acetylglucosaminyltransferase 2), Q9NZV5 (Selenoprotein N), Q9P283 (Semaphorin-5B), Q9UBG0 (C-type mannose receptor 2), Q9UBX1 (Cathepsin F), Q9ULB1 (Neurexin-1), Q9Y2C2 (Uronyl 2-sulfotransferase), Q9Y2C3 (Beta-1,3-galactosyltransferase 5), Q9Y4C5 (Carbohydrate sulfotransferase 2), Q9Y4D7 (Plexin-D1), Q9Y5G0 (Protocadherin gamma-B5), Q9Y5L3 (Ectonucleoside triphosphate diphosphohydrolase 2), Q9Y6C2 (EMILIN-1), Q9Y6M7 (Sodium bicarbonate cotransporter 3), Q9Y6N6 (Laminin subunit gamma-3), Q9Y6Q6 (Tumor necrosis factor receptor superfamily member 11A), , , ,

Cluster 2

A2RU67 (Uncharacterized protein KIAA1467), A2VDJ0 (Transmembrane protein 131-like), A8MWY0 (UPF0577 protein KIAA1324-like), O00299 (Chloride intracellular channel protein 1), O14917 (Protocadherin-17), O75051 (Plexin-A2), O75054 (Immunoglobulin superfamily member 3), O75581 (Low-density lipoprotein receptor-related protein 6), O76082 (Solute carrier family 22 member 5), O94813 (Slit homolog 2 protein; Slit homolog 2 protein N-product; Slit homolog 2 protein C-product), P05067 (Amyloid beta A4 protein; N-APP; Soluble APP-alpha; Soluble APP-beta; C99; Beta-amyloid protein 42; Beta-amyloid protein 40; C83; P3(42); P3(40); C80; Gamma-secretase C-terminal fragment 59; Gamma-secretase C-terminal fragment 57; Gamma-secretase C-terminal fragment 50; C31), P05556 (Integrin beta-1), P08648 (Integrin alpha-5; Integrin alpha-5 heavy chain; Integrin alpha-5 light chain), P09668 (Pro-cathepsin H; Cathepsin H mini chain; Cathepsin H; Cathepsin H heavy chain; Cathepsin H light chain), P30491 (HLA class I histocompatibility antigen, B-53 alpha chain), P10319 (HLA class I histocompatibility antigen, B-58 alpha chain), P30481 (HLA class I histocompatibility antigen, B-44 alpha chain), P12259 (Coagulation factor V; Coagulation factor V heavy chain; Coagulation factor V light chain), P13611 (Versican core protein), P14410 (Sucrase-isomaltase, intestinal; Sucrase; Isomaltase), P16422 (Epithelial cell adhesion molecule), P17301 (Integrin alpha-2), P22607 (Fibroblast growth factor receptor 3), P24821 (Tenascin), P27487 (Dipeptidyl peptidase 4; Dipeptidyl peptidase 4 membrane form; Dipeptidyl peptidase 4 soluble form), P29622 (Kallistatin), P31943 (Heterogeneous nuclear ribonucleoprotein H; Heterogeneous nuclear ribonucleoprotein H, N-terminally processed), P32004 (Neural cell adhesion molecule L1), P48167 (Glycine receptor subunit beta), P48230 (Transmembrane 4 L6 family member 4), P49006 (MARCKS-related protein), P52848 (Bifunctional heparan sulfate N-deacetylase/N-sulfotransferase 1; Heparan sulfate N-deacetylase 1; Heparan sulfate N-sulfotransferase 1), P55259 (Pancreatic secretory granule membrane major glycoprotein GP2), P58107 (Epiplakin), P58397 (A disintegrin and metalloproteinase with thrombospondin motifs 12), P78509 (Reelin), P80108 (Phosphatidylinositol-glycan-specific phospholipase D), P98160 (Basement membrane-specific heparan sulfate proteoglycan core protein; Endorepellin; LG3 peptide), P98164 (Low-density lipoprotein receptor-related protein 2), Q10588 (ADP-ribosyl cyclase 2), Q12913 (Receptor-type tyrosine-protein phosphatase eta), Q13428 (Treacle protein), Q14157 (Ubiquitin-associated protein 2-like), Q14264 (HERV-R_7q21.2 provirus ancestral Env polyprotein; Surface protein; Transmembrane protein), Q14517 (Protocadherin Fat 1; Protocadherin Fat 1, nuclear form), Q14832 (Metabotropic glutamate receptor 3), Q16620 (BDNF/NT-3 growth factors receptor), Q2VWP7 (Protogenin), Q4G148 (Glucoside xylosyltransferase 1), Q58EX2 (Protein sidekick-2), Q5GFL6 (von Willebrand factor A domain-containing protein 2), Q5HYA8 (Meckelin), Q68CR1 (Protein sel-1 homolog 3), Q6N022 (Teneurin-4), Q6NUS6 (Tectonic-3), Q6YBV0 (Proton-coupled amino acid transporter 4), Q7LOX0 (TLR4 interactor with leucine rich repeats), Q7Z407 (CUB and sushi domain-containing protein 3), Q7Z7M0 (Multiple epidermal growth factor-like domains protein 8), Q86XX4 (Extracellular matrix protein FRAS1), Q8N6U8 (G-protein coupled receptor 161), Q8N6Y1 (Protocadherin-20), Q8NAT1 (Glycosyltransferase-like domain-containing protein 2), Q8NDV1 (Alpha-N-acetylgalactosaminide alpha-2,6-sialyltransferase 3), Q8NFZ3 (Neuroligin-4, Y-linked), Q8NFZ8 (Cell adhesion molecule 4), Q8TCT7 (Signal peptide peptidase-like 2B), Q8TE59 (A disintegrin and metalloproteinase with thrombospondin motifs 19), Q8WXG9 (G-protein coupled receptor 98), Q8WY21 (VPS10 domain-containing receptor SorCS1), Q9UPU3 (VPS10 domain-containing receptor SorCS3), Q8WY21 (VPS10 domain-containing receptor SorCS1), Q8WZ71 (Transmembrane protein 158), Q92626 (Peroxidasin homolog), Q92729 (Receptor-type tyrosine-protein phosphatase U), Q92823 (Neuronal cell adhesion molecule), Q96MM7 (Heparan-sulfate 6-O-sulfotransferase 2), Q99523 (Sortilin), Q9BTNO (Leucine-rich repeat and fibronectin type-III domain-containing protein 3), Q9BV10 (Dol-P-Man:Man(7)GlcNAc(2)-PP-Dol alpha-1,6-mannosyltransferase), Q9H6X2 (Anthrax toxin receptor 1), Q9P2N4 (A disintegrin and metalloproteinase with thrombospondin motifs 9), Q9UHN6 (Transmembrane protein 2), Q9UK12 (Cdc42 effector protein 3), Q9UL13 (Protein HEG homolog 1), Q9Y2U8 (Inner nuclear membrane protein Mani), Q9Y6X5 (Bis(5′-adenosyl)-triphosphatase ENPP4)

Cluster 3

A2VDJ0 (Transmembrane protein 131-like), A6NE02 (BTB/POZ domain-containing protein 17), A6NH11 (Glycolipid transfer protein domain-containing protein 2), A8MVW5 (HEPACAM family member 2), O00451 (GDNF family receptor alpha-2), O00533 (Neural cell adhesion molecule L1-like protein; Processed neural cell adhesion molecule L1-like protein), O15230 (Laminin subunit alpha-5), O43451 (Maltase-glucoamylase, intestinal; Maltase; Glucoamylase), O60635 (Tetraspanin-1), O75093 (Slit homolog 1 protein), O94769 (Extracellular matrix protein 2), O94856 (Neurofascin), O95477 (ATP-binding cassette sub-family A member 1), P01009 (Alpha-1-antitrypsin; Short peptide from AAT), P07204 (Thrombomodulin), P07307 (Asialoglycoprotein receptor 2), P07602 (Proactivator polypeptide; Saposin-A; Saposin-B-Val; Saposin-B; Saposin-C; Saposin-D), P10643 (Complement component C7), P12107 (Collagen alpha-1(XI) chain), P12821 (Angiotensin-converting enzyme; Angiotensin-converting enzyme, soluble form), P14410 (Sucrase-isomaltase, intestinal; Sucrase; Isomaltase), P15586 (N-acetylglucosamine-6-sulfatase), P16278 (Beta-galactosidase), P16444 (Dipeptidase 1), P16519 (Neuroendocrine convertase 2), P16671 (Platelet glycoprotein 4), P18564 (Integrin beta-6), P18850 (Cyclic AMP-dependent transcription factor ATF-6 alpha; Processed cyclic AMP-dependent transcription factor ATF-6 alpha), P27701 (CD82 antigen), P34810 (Macrosialin), P38435 (Vitamin K-dependent gamma-carboxylase), P40189 (Interleukin-6 receptor subunit beta), P42658 (Dipeptidyl aminopeptidase-like protein 6), P54289 (Voltage-dependent calcium channel subunit alpha-2/delta-1; Voltage-dependent calcium channel subunit alpha-2-1; Voltage-dependent calcium channel subunit delta-1), P55289 (Cadherin-12), P63261 (Actin, cytoplasmic 2; Actin, cytoplasmic 2, N-terminally processed), Q01459 (Di-N-acetylchitobiase), Q12864 (Cadherin-17), Q12866 (Tyrosine-protein kinase Mer), Q13201 (Multimerin-1; Platelet glycoprotein Ia*; 155 kDa platelet multimerin), Q13822 (Ectonucleotide pyrophosphatase/phosphodiesterase family member 2), Q14108 (Lysosome membrane protein 2), Q14517 (Protocadherin Fat 1; Protocadherin Fat 1, nuclear form), Q16787 (Laminin subunit alpha-3), Q16819 (Meprin A subunit alpha), Q5T601 (Probable G-protein coupled receptor 110), Q6J4K2 (Sodium/potassium/calcium exchanger 6, mitochondrial), Q6NUS6 (Tectonic-3), Q6QNK2 (Probable G-protein coupled receptor 133), Q6UW56 (All-trans retinoic acid-induced differentiation factor), Q6UXG2 (UPF0577 protein KIAA1324), Q6UY14 (ADAMTS-like protein 4), Q6Y288 (Beta-1,3-glucosyltransferase), Q75N90 (Fibrillin-3), Q7L985 (Leucine-rich repeat and immunoglobulin-like domain-containing nogo receptor-interacting protein 2), Q7Z3B1 (Neuronal growth regulator 1), Q86SR1 (Polypeptide N-acetylgalactosaminyltransferase 10), Q86Z14 (Beta-klotho), Q81U80 (Transmembrane protease serine 6), Q81WU5 (Extracellular sulfatase Sulf-2), Q81WV2 (Contactin-4), Q81ZP9 (G-protein coupled receptor 64), Q8N3J6 (Cell adhesion molecule 2), Q8N3Z0 (Inactive serine protease 35), Q8N608 (Inactive dipeptidyl peptidase 10), Q8N8Z6 (Discoidin, CUB and LCCL domain-containing protein 1), Q8TDU6 (G-protein coupled bile acid receptor 1), Q8TDW7 (Protocadherin Fat 3), Q8WUT4 (Leucine-rich repeat neuronal protein 4), Q92187 (CMP-N-acetylneuraminate-poly-alpha-2,8-sialyltransferase), Q92508 (Piezo-type mechanosensitive ion channel component 1), Q92629 (Delta-sarcoglycan), Q96FT7 (Acid-sensing ion channel 4), Q96P44 (Collagen alpha-1(XXI) chain), Q96PB7 (Noelin-3), Q99571 (P2X purinoceptor 4), Q9BUJ0 (Alpha/beta hydrolase domain-containing protein 14A), Q9H7M9 (Platelet receptor Gi24), Q9H8M5 (Metal transporter CNNM2), Q9H9S5 (Fukutin-related protein), Q9HCU4 (Cadherin EGF LAG seven-pass G-type receptor 2), Q9NSC7 (Alpha-N-acetylgalactosaminide alpha-2,6-sialyltransferase 1), Q9NT68 (Teneurin-2; Ten-2, soluble form; Ten-2 intracellular domain), Q9NU53 (Glycoprotein integral membrane protein 1), Q9UJ37 (Alpha-N-acetylgalactosaminide alpha-2,6-sialyltransferase 2), Q9UKR0 (Kallikrein-12), Q9Y5F0 (Protocadherin beta-13), Q9Y6M7 (Sodium bicarbonate cotransporter 3)

Cluster 4

O00391 (Sulfhydryl oxidase 1), O00567 (Nucleolar protein 56), O60512 (Beta-1,4-galactosyltransferase 3; N-acetyllactosamine synthase; Beta-N-acetylglucosaminylglycopeptide beta-1,4-galactosyltransferase; Beta-N-acetylglucosaminyl-glycolipid beta-1,4-galactosyltransferase), O75462 (Cytokine receptor-like factor 1), O94898 (Leucine-rich repeats and immunoglobulin-like domains protein 2), O94905 (Erlin-2), O75477 (Erlin-1), O95502 (Neuronal pentraxin receptor), O95813 (Cerberus), O96005 (Cleft lip and palate transmembrane protein 1), P04216 (Thy-1 membrane glycoprotein), P05556 (Integrin beta-1), P10721 (Mast/stem cell growth factor receptor Kit), P11047 (Laminin subunit gamma-1), P11362 (Fibroblast growth factor receptor 1), P13611 (Versican core protein), P17301 (Integrin alpha-2), P21709 (Ephrin type-A receptor 1), P21802 (Fibroblast growth factor receptor 2), P24821 (Tenascin), P25189 (Myelin protein P0), P32004 (Neural cell adhesion molecule L1), P43121 (Cell surface glycoprotein MUC18), P52849 (Bifunctional heparan sulfate N-deacetylase/N-sulfotransferase 2; Heparan sulfate N-deacetylase 2; Heparan sulfate N-sulfotransferase 2), P56199 (Integrin alpha-1), P62847 (40S ribosomal protein S24), P78536 (Disintegrin and metalloproteinase domain-containing protein 17), P98153 (Integral membrane protein DGCR2/IDD), Q002388 (Collagen alpha-1(VII) chain), Q007954 (Prolow-density lipoprotein receptor-related protein 1; Low-density lipoprotein receptor-related protein 1 85 kDa subunit; Low-density lipoprotein receptor-related protein 1 515 kDa subunit; Low-density lipoprotein receptor-related protein 1 intracellular domain), Q10472 (Polypeptide N-acetylgalactosaminyltransferase 1; Polypeptide N-acetylgalactosaminyltransferase 1 soluble form), Q12913 (Receptor-type tyrosine-protein phosphatase eta), Q13275 (Semaphorin-3F), Q14517 (Protocadherin Fat 1; Protocadherin Fat 1, nuclear form), Q14982 (Opioid-binding protein/cell adhesion molecule), Q15904 (V-type proton ATPase subunit Si), Q3T906 (N-acetylglucosamine-1-phosphotransferase subunits alpha/beta; N-acetylglucosamine-1-phosphotransferase subunit alpha; N-acetylglucosamine-1-phosphotransferase subunit beta), Q4KMQ2 (Anoctamin-6), Q5KU26 (Collectin-12), Q61S24 (Putative polypeptide N-acetylgalactosaminyltransferase-like protein 3), Q6NSJ0 (Uncharacterized family 31 glucosidase KIAA1161), Q6YBV0 (Proton-coupled amino acid transporter 4), Q6ZQN7 (Solute carrier organic anion transporter family member 4C1), Q6ZRP7 (Sulfhydryl oxidase 2), Q86TL2 (Transmembrane protein 110), Q86UF1 (Tetraspanin-33), Q86WK7 (Amphoterin-induced protein 3), Q86XX4 (Extracellular matrix protein FRAS1), Q81ZA0 (Dyslexia-associated protein KIAA0319-like protein), Q8NOZ9 (V-set and immunoglobulin domain-containing protein 10), Q8N8Z6 (Discoidin, CUB and LCCL domain-containing protein 1), Q8NDV1 (Alpha-N-acetylgalactosaminide alpha-2,6-sialyltransferase 3), Q8TE58 (A disintegrin and metalloproteinase with thrombospondin motifs 15), Q92673 (Sortilin-related receptor), Q92729 (Receptor-type tyrosine-protein phosphatase U), Q92896 (Golgi apparatus protein 1), Q93063 (Exostosin-2), Q969N2 (GPI transamidase component PIG-T), Q96JA1 (Leucine-rich repeats and immunoglobulin-like domains protein 1), Q96KA5 (Cleft lip and palate transmembrane protein 1-like protein), Q96MK3 (Protein FAM20A), Q9BRK3 (Matrix-remodeling-associated protein 8), Q9BXB1 (Leucine-rich repeat-containing G-protein coupled receptor 4), Q9BZR6 (Reticulon-4 receptor), Q9COC4 (Semaphorin-4C), Q9H0X4 (Protein ITFG3), Q9H497 (Torsin-3A), Q9H5J4 (Elongation of very long chain fatty acids protein 6), Q9H813 (Transmembrane protein 206), Q9NWD8 (Transmembrane protein 248), Q9P2W7 (Galactosylgalactosylxylosylprotein 3-beta-glucuronosyltransferase 1), Q9UIQ6 (Leucyl-cystinyl aminopeptidase; Leucyl-cystinyl aminopeptidase, pregnancy serum form), Q9ULF5 (Zinc transporter ZIP10), Q9UN73 (Protocadherin alpha-6), Q9Y4K0 (Lysyl oxidase homolog 2), Q9Y6L7 (Tolloid-like protein 2), Q9Y6X5 (Bis(5′-adenosyl)-triphosphatase ENPP4)

Cluster 5

A8MWY0 (UPF0577 protein KIAA1324-like), O00754 (Lysosomal alpha-mannosidase; Lysosomal alpha-mannosidase A peptide; Lysosomal alpha-mannosidase B peptide; Lysosomal alpha-mannosidase C peptide; Lysosomal alpha-mannosidase D peptide; Lysosomal alpha-mannosidase E peptide), O14786 (Neuropilin-1), O14917 (Protocadherin-17), P19105 (Myosin regulatory light chain 12A), O14950 (Myosin regulatory light chain 12B), P24844 (Myosin regulatory light polypeptide 9), O15123 (Angiopoietin-2), O15230 (Laminin subunit alpha-5), O43451 (Maltase-glucoamylase, intestinal; Maltase; Glucoamylase), O60245 (Protocadherin-7), O60449 (Lymphocyte antigen 75), O60486 (Plexin-C1), O60494 (Cubilin), O60512 (Beta-1,4-galactosyltransferase 3; N-acetyllactosamine synthase; Beta-N-acetylglucosam inylg lycopeptide beta-1,4-galactosyltransferase; Beta-N-acetylglucosaminyl-glycolipid beta-1,4-galactosyltransferase), O75054 (Immunoglobulin superfamily member 3), O75071 (EF-hand calcium-binding domain-containing protein 14), O75094 (Slit homolog 3 protein), O75129 (Astrotactin-2), O75356 (Ectonucleoside triphosphate diphosphohydrolase 5), O75503 (Ceroid-lipofuscinosis neuronal protein 5), O75752 (UDP-GalNAc:beta-1,3-N-acetylgalactosaminyltransferase 1), O75882 (Attractin), O76024 (Wolframin), O76082 (Solute carrier family 22 member 5), Q9H015 (Solute carrier family 22 member 4), O94923 (D-glucuronyl C5-epimerase), O95084 (Serine protease 23), O95477 (ATP-binding cassette sub-family A member 1), O95858 (Tetraspanin-15), P00533 (Epidermal growth factor receptor), P00734 (Prothrombin; Activation peptide fragment 1; Activation peptide fragment 2; Thrombin light chain; Thrombin heavy chain), P01009 (Alpha-1-antitrypsin; Short peptide from AAT), P01011 (Alpha-1-antichymotrypsin; Alpha-1-antichymotrypsin His-Pro-less), P01857 (Ig gamma-1 chain C region), P02458 (Collagen alpha-1(II) chain; Collagen alpha-1(II) chain; Chondrocalcin), P02675 (Fibrinogen beta chain; Fibrinopeptide B; Fibrinogen beta chain), P02679 (Fibrinogen gamma chain), P02771 (Alpha-fetoprotein), P02787 (Serotransferrin), P02790 (Hemopexin), P04004 (Vitronectin; Vitronectin V65 subunit; Vitronectin V10 subunit; Somatomedin-B), P04066 (Tissue alpha-L-fucosidase), P04114 (Apolipoprotein B-100; Apolipoprotein B-48), P05154 (Plasma serine protease inhibitor), P05556 (Integrin beta-1), P06213 (Insulin receptor; Insulin receptor subunit alpha; Insulin receptor subunit beta), P06276 (Cholinesterase), P07306 (Asialoglycoprotein receptor 1), P07858 (Cathepsin B; Cathepsin B light chain; Cathepsin B heavy chain), P07942 (Laminin subunit beta-1), P07996 (Thrombospondin-1), P08842 (Steryl-sulfatase), P09758 (Tumor-associated calcium signal transducer 2), P10253 (Lysosomal alpha-glucosidase; 76 kDa lysosomal alpha-glucosidase; 70 kDa lysosomal alpha-glucosidase), P10645 (Chromogranin-A; Vasostatin-1; Vasostatin-2; EA-92; ES-43; Pancreastatin; SS-18; WA-8; WE-14; LF-19; AL-11; GV-19; GR-44; ER-37), P10909 (Clusterin; Clusterin beta chain; Clusterin alpha chain), P12821 (Angiotensin-converting enzyme; Angiotensin-converting enzyme, soluble form), P13591 (Neural cell adhesion molecule 1), P14314 (Glucosidase 2 subunit beta), P14384 (Carboxypeptidase M), P14410 (Sucrase-isomaltase, intestinal; Sucrase; Isomaltase), P15586 (N-acetylglucosamine-6-sulfatase), P16144 (Integrin beta-4), P16234 (Platelet-derived growth factor receptor alpha), P16278 (Beta-galactosidase), P16444 (Dipeptidase 1), P17936 (Insulin-like growth factor-binding protein 3), P21589 (5′-nucleotidase), P22304 (Iduronate 2-sulfatase; Iduronate 2-sulfatase 42 kDa chain; Iduronate 2-sulfatase 14 kDa chain), P23142 (Fibulin-1), P23276 (Kell blood group glycoprotein), P25391 (Laminin subunit alpha-1), P25929 (Neuropeptide Y receptor type 1), P26006 (Integrin alpha-3; Integrin alpha-3 heavy chain; Integrin alpha-3 light chain), P26012 (Integrin beta-8), P27487 (Dipeptidyl peptidase 4; Dipeptidyl peptidase 4 membrane form; Dipeptidyl peptidase 4 soluble form), P28827 (Receptor-type tyrosine-protein phosphatase mu), P34059 (N-acetylgalactosamine-6-sulfatase), P35475 (Alpha-L-iduronidase), P35555 (Fibrillin-1), P36578 (60S ribosomal protein L4), P42785 (Lysosomal Pro-X carboxypeptidase), P43146 (Netrin receptor DCC), P48357 (Leptin receptor), P51689 (Arylsulfatase D), P51690 (Arylsulfatase E), P51805 (Plexin-A3), P52799 (Ephrin-B2), P52848 (Bifunctional heparan sulfate N-deacetylase/N-sulfotransferase 1; Heparan sulfate N-deacetylase 1; Heparan sulfate N-sulfotransferase 1), P54289 (Voltage-dependent calcium channel subunit alpha-2/delta-1; Voltage-dependent calcium channel subunit alpha-2-1; Voltage-dependent calcium channel subunit delta-1), P54753 (Ephrin type-B receptor 3), P55259 (Pancreatic secretory granule membrane major glycoprotein GP2), P55268 (Laminin subunit beta-2), P55285 (Cadherin-6), P55899 (IgG receptor FcRn large subunit p51), P56817 (Beta-secretase 1), P58397 (A disintegrin and metalloproteinase with thrombospondin motifs 12), P60709 (Actin, cytoplasmic 1; Actin, cytoplasmic 1, N-terminally processed), P61812 (Transforming growth factor beta-2; Latency-associated peptide), P78509 (Reelin), P98160 (Basement membrane-specific heparan sulfate proteoglycan core protein; Endorepellin; LG3 peptide), P98164 (Low-density lipoprotein receptor-related protein 2), Q08334 (Interleukin-10 receptor subunit beta), Q12864 (Cadherin-17), Q13253 (Noggin), Q13433 (Zinc transporter ZIP6), Q13873 (Bone morphogenetic protein receptor type-2), Q14112 (Nidogen-2), Q14126 (Desmoglein-2), Q14393 (Growth arrest-specific protein 6), Q14517 (Protocadherin Fat 1; Protocadherin Fat 1, nuclear form), Q14623 (Indian hedgehog protein; Indian hedgehog protein N-product; Indian hedgehog protein C-product), Q14643 (Inositol 1,4,5-trisphosphate receptor type 1), Q24JP5 (Transmembrane protein 132A), Q2MV58 (Tectonic-1), Q2VWP7 (Protogenin), Q3MIR4 (Cell cycle control protein 50B), Q4ZIN3 (Membralin), Q504Y2 (Protein kinase domain-containing protein, cytoplasmic), Q53RD9 (Fibulin-7), Q5H8A4 (GPI ethanolamine phosphate transferase 2), Q5H8C1 (FRAS1-related extracellular matrix protein 1), Q5JS37 (NHL repeat-containing protein 3), Q5SZK8 (FRAS1-related extracellular matrix protein 2), Q5T4D3 (Transmembrane and TPR repeat-containing protein 4), Q68CP4 (Heparan-alpha-glucosaminide N-acetyltransferase), Q6L9W6 (Beta-1,4-N-acetylgalactosaminyltransferase 3), Q6N022 (Teneurin-4), Q6NSJ0 (Uncharacterized family 31 glucosidase KIAA1161), Q6NUS8 (UDP-glucuronosyltransferase 3A1), Q6PK18 (2-oxoglutarate and iron-dependent oxygenase domain-containing protein 3), Q6PKC3 (Thioredoxin domain-containing protein 11), Q6QNK2 (Probable G-protein coupled receptor 133), Q6UWL2 (Sushi domain-containing protein 1), Q6UXG2 (UPF0577 protein KIAA1324), Q6UXK5 (Leucine-rich repeat neuronal protein 1), Q6UXM1 (Leucine-rich repeats and immunoglobulin-like domains protein 3), Q6W3E5 (Glycerophosphodiester phosphodiesterase domain-containing protein 4), Q6ZTQ4 (Cadherin-related family member 3), Q7L112 (Synaptic vesicle glycoprotein 2B), Q7L1S5 (Carbohydrate sulfotransferase 9), Q7Z5N4 (Protein sidekick-1), Q7Z739 (YTH domain family protein 3), Q7Z7M9 (Polypeptide N-acetylgalactosaminyltransferase 5), Q7Z7N9 (Transmembrane protein 179B), Q86SQ4 (G-protein coupled receptor 126), Q86SR1 (Polypeptide N-acetylgalactosaminyltransferase 10), Q86VZ4 (Low-density lipoprotein receptor-related protein 11), Q86WK6 (Amphoterin-induced protein 1), Q86XK7 (V-set and immunoglobulin domain-containing protein 1), Q81UX7 (Adipocyte enhancer-binding protein 1), Q81WD5 (Major facilitator superfamily domain-containing protein 6-like), Q81WK6 (Probable G-protein coupled receptor 125), Q81WV2 (Contactin-4), Q81X30 (Signal peptide, CUB and EGF-like domain-containing protein 3), Q81XL6 (Extracellular serine/threonine protein kinase FAM20C), Q81ZJ3 (C3 and PZP-like alpha-2-macroglobulin domain-containing protein 8), Q8N0V4 (Leucine-rich repeat LGI family member 2), Q8N3J6 (Cell adhesion molecule 2), Q8N608 (Inactive dipeptidyl peptidase 10), Q8NB15 (Solute carrier family 43 member 3), Q8NBL3 (Transmembrane protein 178A), Q8NCR0 (UDP-GalNAc:beta-1,3-N-acetylgalactosaminyltransferase 2), Q8NE01 (Metal transporter CNNM3), Q9H8M5 (Metal transporter CNNM2), Q8NE01 (Metal transporter CNNM3), Q8NES3 (Beta-1,3-N-acetylglucosaminyltransferase lunatic fringe), Q8NFZ8 (Cell adhesion molecule 4), Q8TE57 (A disintegrin and metalloproteinase with thrombospondin motifs 16), Q8WTR4 (Glycerophosphodiester phosphodiesterase domain-containing protein 5), Q8WUT4 (Leucine-rich repeat neuronal protein 4), Q8WVJ2 (NudC domain-containing protein 2), Q8WW52 (Protein FAM151A), Q8WXG9 (G-protein coupled receptor 98), Q92484 (Acid sphingomyelinase-like phosphodiesterase 3a), Q92823 (Neuronal cell adhesion molecule), Q92896 (Golgi apparatus protein 1), Q92932 (Receptor-type tyrosine-protein phosphatase N2), Q96F46 (Interleukin-17 receptor A), Q96GX1 (Tectonic-2), Q96JQ0 (Protocadherin-16), Q96L58 (Beta-1,3-galactosyltransferase 6), Q96SM3 (Probable carboxypeptidase X1), Q99519 (Sialidase-1), Q99985 (Semaphorin-3C), Q9BRN9 (TM2 domain-containing protein 3), Q9BXX0 (EMILIN-2), Q9BYH1 (Seizure 6-like protein), Q9COH2 (Protein tweety homolog 3), Q9H156 (SLIT and NTRK-like protein 2), Q9H3T3 (Semaphorin-6B), Q9H488 (GDP-fucose protein O-fucosyltransferase 1), Q9H4F8 (SPARC-related modular calcium-binding protein 1), Q9HAT2 (Sialate O-acetylesterase), Q9HBW1 (Leucine-rich repeat-containing protein 4), Q9HC56 (Protocadherin-9), Q9HCK4 (Roundabout homolog 2), Q9HCU4 (Cadherin EGF LAG seven-pass G-type receptor 2), Q9NPH3 (Interleukin-1 receptor accessory protein), Q9NPR2 (Semaphorin-4B), Q9NQ84 (G-protein coupled receptor family C group 5 member C), Q9NR99 (Matrix-remodeling-associated protein 5), Q9NTN9 (Semaphorin-4G), Q9NY72 (Sodium channel subunit beta-3), Q9NZU0 (Leucine-rich repeat transmembrane protein FLRT3), Q9P232 (Contactin-3), Q9P2B2 (Prostaglandin F2 receptor negative regulator), Q9P2N4 (A disintegrin and metalloproteinase with thrombospondin motifs 9), Q9UBP4 (Dickkopf-related protein 3), Q9UHC9 (Niemann-Pick C1-like protein 1), Q9UIQ6 (Leucyl-cystinyl aminopeptidase; Leucyl-cystinyl aminopeptidase, pregnancy serum form), Q9UKP4 (A disintegrin and metalloproteinase with thrombospondin motifs 7), Q9UKU9 (Angiopoietin-related protein 2), Q9UMR5 (Lysosomal thioesterase PPT2), Q9UQB3 (Catenin delta-2), Q9Y2E5 (Epididymis-specific alpha-mannosidase), Q9Y2G1 (Myelin regulatory factor), Q9Y2G5 (GDP-fucose protein O-fucosyltransferase 2), Q9Y5E4 (Protocadherin beta-5), Q9Y5F9 (Protocadherin gamma-B6), Q9Y5H9 (Protocadherin alpha-2), Q9Y512 (Protocadherin alpha-10), Q9Y5X9 (Endothelial lipase), Q9Y5Y6 (Suppressor of tumorigenicity 14 protein), Q9Y6N7 (Roundabout homolog 1), Q9Y6N8 (Cadherin-10), Q9Y6X5 (Bis(5′-adenosyl)-triphosphatase ENPP4)

Cluster 6

O00584 (Ribonuclease T2), O15230 (Laminin subunit alpha-5), O43490 (Prominin-1), O43567 (E3 ubiquitin-protein ligase RNF13), O60486 (Plexin-C1), O75071 (EF-hand calcium-binding domain-containing protein 14), O94910 (Latrophilin-1), P00749 (Urokinase-type plasminogen activator; Urokinase-type plasminogen activator long chain A; Urokinase-type plasminogen activator short chain A; Urokinase-type plasminogen activator chain B), P04626 (Receptor tyrosine-protein kinase erbB-2), P09486 (SPARC), P0C7U0 (Protein ELFN1), P12109 (Collagen alpha-1(VI) chain), P12110 (Collagen alpha-2(VI) chain), P13688 (Carcinoembryonic antigen-related cell adhesion molecule 1), P15309 (Prostatic acid phosphatase; PAPf39), P16070 (CD44 antigen), P16422 (Epithelial cell adhesion molecule), P16671 (Platelet glycoprotein 4), P23468 (Receptor-type tyrosine-protein phosphatase delta), P48960 (CD97 antigen; CD97 antigen subunit alpha; CD97 antigen subunit beta), P55290 (Cadherin-13), P57087 (Junctional adhesion molecule B), Q05707 (Collagen alpha-1(XIV) chain), Q06418 (Tyrosine-protein kinase receptor TYRO3), Q06430 (N-acetyllactosaminide beta-1,6-N-acetylglucosaminyl-transferase, isoform B), Q8NOV5 (N-acetyllactosaminide beta-1,6-N-acetylglucosaminyl-transferase, isoform A), Q07075 (Glutamyl aminopeptidase), Q13797 (Integrin alpha-9), Q14118 (Dystroglycan; Alpha-dystroglycan; Beta-dystroglycan), Q14563 (Semaphorin-3A), Q16769 (Glutaminyl-peptide cyclotransferase), Q16849 (Receptor-type tyrosine-protein phosphatase-like N), Q3SY77 (UDP-glucuronosyltransferase 3A2), Q53EL9 (Seizure protein 6 homolog), Q51J48 (Protein crumbs homolog 2), Q5VSYO (G kinase-anchoring protein 1), Q6AZY7 (Scavenger receptor class A member 3), Q6UWJ1 (Transmembrane and coiled-coil domain-containing protein 3), Q6UWL2 (Sushi domain-containing protein 1), Q6UXK5 (Leucine-rich repeat neuronal protein 1), Q6ZRP7 (Sulfhydryl oxidase 2), Q86WC4 (Osteopetrosis-associated transmembrane protein 1), Q86WK7 (Amphoterin-induced protein 3), Q81W45 (ATP-dependent (S)-NAD(P)H-hydrate dehydratase), Q81XL6 (Extracellular serine/threonine protein kinase FAM20C), Q8N5B7 (Ceramide synthase 5), Q8N6Q3 (CD177 antigen), Q8NFLO (UDP-GlcNAc:betaGal beta-1,3-N-acetylglucosaminyltransferase 7), Q8NFS9 (N-acetyllactosaminide beta-1,6-N-acetylglucosaminyl-transferase, isoform C), Q8NFT8 (Delta and Notch-like epidermal growth factor-related receptor), Q8TDW7 (Protocadherin Fat 3), Q8TE99 (Acid phosphatase-like protein 2), Q8WXG9 (G-protein coupled receptor 98), Q92692 (Poliovirus receptor-related protein 2), Q96BA8 (Cyclic AMP-responsive element-binding protein 3-like protein 1; Processed cyclic AMP-responsive element-binding protein 3-like protein 1), Q99784 (Noelin), Q9BXB1 (Leucine-rich repeat-containing G-protein coupled receptor 4), Q9GZX3 (Carbohydrate sulfotransferase 6), Q9HCC8 (Glycerophosphoinositol inositolphosphodiesterase GDPD2), Q9NPF2 (Carbohydrate sulfotransferase 11), Q9NRB3 (Carbohydrate sulfotransferase 12), Q9Y251 (Heparanase; Heparanase 8 kDa subunit; Heparanase 50 kDa subunit), Q9Y2C2 (Uronyl 2-sulfotransferase), Q9Y3Q0 (N-acetylated-alpha-linked acidic dipeptidase 2), Q9Y653 (G-protein coupled receptor 56), GPR56 (N-terminal fragment), GPR56 (C-terminal fragment),

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

### REFERENCE LIST

1 Cogger, K. & Nostro, M. C. Recent advances in cell replacement therapies for the treatment of type 1 diabetes. Endocrinology 156, 8-15, doi:10.1210/en.2014-1691 (2015).
2 Vegas, A. J. et al. Long-term glycemic control using polymer-encapsulated human stem cell-derived beta cells in immune-competent mice. Nat Med 22, 306-311, doi:10.1038/nm.4030 (2016).
3 Agulnick, A. D. et al. Insulin-Producing Endocrine Cells Differentiated In Vitro From Human Embryonic Stem Cells Function in Macroencapsulation Devices In Vivo. Stem Cells Transl Med 4, 1214-1222, doi:10.5966/sctm.2015-0079 (2015).
4 Bruin, J. E. et al. Maturation and function of human embryonic stem cell-derived pancreatic progenitors in macroencapsulation devices following transplant into mice. Diabetologia 56, 1987-1998, doi:10.1007/s00125-013-2955-4 (2013).
5 Szot, G. L. et al. Tolerance induction and reversal of diabetes in mice transplanted with human embryonic stem cell-derived pancreatic endoderm. Cell Stem Cell 16, 148-157, doi:10.1016/j.stem.2014.12.001 (2015).
6 Cho, C. H. et al. Inhibition of activin/nodal signalling is necessary for pancreatic differentiation of human pluripotent stem cells. Diabetologia 55, 3284-3295, doi:10.1007/s00125-012-2687-x (2012).
7 Kelly, O. G. et al. Cell-surface markers for the isolation of pancreatic cell types derived from human embryonic stem cells. Nat Biotechnol 29, 750-756 (2011).
8 Kroon, E. et al. Pancreatic endoderm derived from human embryonic stem cells generates glucose-responsive insulin-secreting cells in vivo. Nat Biotechnol 26, 443-452 (2008).
9 Nostro, M. C. et al. Efficient generation of NKX6-1+ pancreatic progenitors from multiple human pluripotent stem cell lines. Stem cell reports 4, 591-604, doi:10.1016/j.stemcr.2015.02.017 (2015).
Pagliuca, F. W. et al. Generation of Functional Human Pancreatic beta Cells In Vitro. Cell 159, 428-439, doi:10.1016/j.cell.2014.09.040 (2014).
11 Rezania, A. et al. Reversal of diabetes with insulin-producing cells derived in vitro from human pluripotent stem cells. Nat Biotechnol, doi:10.1038/nbt.3033 (2014).
12 Rezania, A. et al. Maturation of human embryonic stem cell-derived pancreatic progenitors into functional islets capable of treating pre-existing diabetes in mice. Diabetes 61, 2016-2029, doi:1 0.2337/dbl 1-1711 (2012).
13 Rezania, A. et al. Enrichment of human embryonic stem cell-derived NKX6.1-expressing pancreatic progenitor cells accelerates the maturation of insulin-secreting cells in vivo. Stem Cells, doi:10.1002/stem.1489 (2013).
14 Russ, H. A. et al. Controlled induction of human pancreatic progenitors produces functional beta-like cells in vitro. EMBO J 34, 1759-1772, doi:10.15252/embj.201591058 (2015).
Korytnikov, R. & Nostro, M. C. Generation of polyhormonal and multipotent pancreatic progenitor lineages from human pluripotent stem cells. Methods 101, 56-64, doi:10.1016/j.ymeth.2015.10.017 (2016).
16 Rezania, A. et al. Enrichment of human embryonic stem cell-derived NKX6.1-expressing pancreatic progenitor cells accelerates the maturation of insulin-secreting cells in vivo. Stem Cells 31, 2432-2442, doi:10.1002/stem.1489 (2013).
17 Basford, C. L. et al. The functional and molecular characterisation of human embryonic stem cell-derived insulin-positive cells compared with adult pancreatic beta cells. Diabetologia 55, 358-371, doi:10.1007/s001 25-011-2335-x (2012).
18 Evseenko, D. et al. Mapping the first stages of mesoderm commitment during differentiation of human embryonic stem cells. Proc Natl Acad Sci USA 107, 13742-13747, doi:10.1073/pnas.1002077107 (2010).
19 Boheler, K. R. et al. A human pluripotent stem cell surface N-glycoproteome resource reveals markers, extracellular epitopes, and drug targets. Stem cell reports 3, 185-203, doi:10.1016/j.stemcr.2014.05.002 (2014).
20 Schaffer, A. E., Freude, K. K., Nelson, S. B. & Sander, M. Nkx6 transcription factors and Ptfl a function as antagonistic lineage determinants in multipotent pancreatic progenitors. Dev Cell 18, 1022-1029, doi:1 0.1016/j.devcel.2010.05.015 (2010).
21 Zhou, Q. et al. A multipotent progenitor domain guides pancreatic organogenesis. Dev Cell 13, 103-114 (2007).
22 Dorrell, C. et al. Isolation of major pancreatic cell types and long-term culture-initiating cells using novel human surface markers. Stem Cell Res 1, 183-194 (2008).
23 Gu, G., Dubauskaite, J. & Melton, D. A. Direct evidence for the pancreatic lineage: NGN3+ cells are islet progenitors and are distinct from duct progenitors. Development 129, 2447-2457 (2002).
24 Solar, M. et al. Pancreatic exocrine duct cells give rise to insulin-producing beta cells during embryogenesis but not after birth. Dev Cell 17, 849-860, doi:10.1016/j.devcel.2009.11.003 (2009).
25 MacDonald, R. J. & Ronzio, R. A. Comparative analysis of zymogen granule membrane polypeptides. Biochem Biophys Res Commun 49, 377-382 (1972).
26 Werner, L. et al. Identification of pancreatic glycoprotein 2 as an endogenous immunomodulator of innate and adaptive immune responses. J Immunol 189, 2774-2783, doi:1 0.4049/jimmunol.1 103190 (2012).
27 Yu, S., Michie, S. A. & Lowe, A. W. Absence of the major zymogen granule membrane protein, GP2, does not affect pancreatic morphology or secretion. J Biol Chem 279, 50274-50279, doi:10.1074/jbc.M410599200 (2004).
28 Cebola, I. et al. TEAD and YAP regulate the enhancer network of human embryonic pancreatic progenitors. Nat Cell Biol 17, 615-626, doi:1 0.1038/ncb3160 (2015).
29 Rugg-Gunn, P. J. et al. Cell-surface proteomics identifies lineage-specific markers of embryo-derived stem cells. Dev Cell 22, 887-901, doi:10.1016/j.devcel.2012.01.005 (2012).
30 Witty, A. D. et al. Generation of the epicardial lineage from human pluripotent stem cells. Nat Biotechnol 32, 1026-1035, doi:10.1038/nbt.3002 (2014).
31 Kennedy, M., D'Souza, S. L., Lynch-Kattman, M., Schwantz, S. & Keller, G. Development of the hemangioblast defines the onset of hematopoiesis in human ES cell differentiation cultures. Blood 109, 2679-2687 (2007).
32 Tian, Y., Zhou, Y., Elliott, S., Aebersold, R. & Zhang, H. Solid-phase extraction of N-linked glycopeptides. Nat Protoc 2, 334-339, doi:10.1038/nprot.2007.42 (2007).
33 Sinha, A., Ignatchenko, V., Ignatchenko, A., Mejia-Guerrero, S. & Kislinger, T. In-depth proteomic analyses of ovarian cancer cell line exosomes reveals differential enrichment of functional categories compared to the NCI 60 proteome. Biochem Biophys Res Commun 445, 694-701, doi:1 0.1016/j.bbrc.2013.12.070 (2014).
34 Cox, J. & Mann, M. MaxQuant enables high peptide identification rates, individualized p.p.b.-range mass accuracies and proteome-wide protein quantification. Nat Biotechnol 26, 1367-1372, doi:10.1038/nbt.1511 (2008).
35 Ameri, J. et al. Efficient Generation of Glucose-Responsive Beta Cells from Isolated GP2+ Human Pancreatic Progenitors. Cell Rep 19, 36-49, doi:10.1016/j.celrep.2017.03.032 (2017).

1. A method for enriching/purifying a population of cells for pancreatic progenitor cells, the method comprising:
a. providing the population cells, the population comprising pancreatic progenitor cells;
b. performing at least one of steps (i)-(iv):
i. selecting for cells from the population that express at least one protein listed in cluster 2;
ii. selecting for cells from the population that express at least one protein listed in cluster 5;
iii. deselecting for cells from the population that express at least one protein listed in cluster 1;
iv. deselecting for cells from the population that express at least one protein listed in cluster 3; and
v. deselecting for cells from the population that express at least one protein listed in cluster 6.

2. The method of claim 1, wherein the at least one protein listed in cluster 2 is any number from 2 to u, wherein u is the total number of proteins listed in cluster 2.

3. The method of claim 1, wherein the at least one protein listed in cluster 5 is any number from 2 to w, wherein w is the total number of proteins listed in cluster 5.

4. The method of claim 1, wherein the at least one protein listed in cluster 1 is any number from 2 to x, wherein w is the total number of proteins listed in cluster 1.

5. The method of claim 1, wherein the at least one protein listed in cluster 3 is any number from 2 to y, wherein w is the total number of proteins listed in cluster 3.

6. The method of claim 1, wherein the at least one protein listed in cluster 6 is any number from 2 to z, wherein w is the total number of proteins listed in cluster 6.

7. The method of claim 1, wherein the at least one step is at least 2 steps.

8. The method of claim 1, wherein the at least one step is at least 3 steps.

9. The method of claim 1, wherein the at least one step is at least 4 steps.

10. The method of claim 1, wherein the at least one step is at least 5 steps.

11. The method of claim 1, wherein the pancreatic progenitor cells are PDX1⁺/NKX6-1⁺.

12. The method of claim 1, wherein the population further comprises at least one of human embryonic stem cells, human pluripotent stem cells, and polyhormonal cells.

13. The method of claim 12, wherein the population further comprises both human embryonic stem cells, and polyhormonal cells.

14. The method of claim 1, wherein the population had been induced to at least partially differentiate into pancreatic progenitor cells from human pluripotent stem cells.

15. The method of claim 1, wherein the selection or deselection step (i), (ii), (iii), (iv) or (v) are performed using an antibody against the at least one protein.

16. The method of claim 15, wherein the antibody is bound to a support.

17. The method of claim 15, wherein the antibody is bound to a fluorophore

18. The method of claim 15, wherein the antibody is bound to a magnetic bead.

19. The method of claim 1, wherein the selection or deselection step is performed using FACS.

20. The method of claim 1, wherein the selection or deselection step is performed using MACS.

21. A kit comprising a plurality of antibodies against a plurality of proteins from at least one of cluster 1, 2, 3, 5, 6, and combinations thereof.

22. The kit of claim 19, wherein the plurality of antibodies are bound to magnetic beads.

23. The kit of claim 19, the plurality of proteins is any number between 2 and q, wherein q is independently the total number of proteins listed in a particular cluster.
